(19) United States
(12) Patent Application Publication
(10) Pub. No.: US 2005/0251029 A1
(43) Pub. Date: Nov. 10, 2005

(54) Radiation therapy treatment plan

(76) Inventors: Ali Khamene, Princeton, NJ (US); Frank Sauer, Princeton, NJ (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/110,156

(22) Filed: Apr. 20, 2005

Related U.S. Application Data
(60) Provisional application No. 60/564,147, filed on Apr. 21, 2004.

(51) Int. Cl. A61N 5/10; G06K 9/00; A61B 5/05

(52) U.S. Cl. 600/427; 382/132

(57) ABSTRACT

A method and system for modifying a radiation therapy treatment plan are disclosed. In one embodiment, a radiation therapy treatment plan based on a first medical diagnostic image of a target in a patient is provided. A second medical diagnostic image of the target is generated while the patient is disposed on a treatment surface of a radiation therapy device. A deformation of the target is determined using the second medical diagnostic image, and the radiation therapy treatment plan is modified based on the determined deformation. Other embodiments are provided, and each of the embodiments described herein can be used alone or in combination with one another.

### CROSS-REFERENCE TO RELATED APPLICATION

This application claims the benefit of U.S. Provisional Application No. 60/564,147, filed Apr. 21, 2004, which is hereby incorporated by reference.

### BACKGROUND

Three-dimensional treatment planning systems accommodate the acquisition and display of three-dimensional patient and dose data. Treatment planning systems are tools used by physicians, physicists, and dosimetrists to plan and calculate doses to the patient. They feature various methods of acquiring patient data. In modern three-dimensional planning systems, the form of patient data is usually a computed tomography (CT) scan. MR images (MRI) or positron emission tomography (PET) images may also be used in conjunction with CT images. Once the patient data is in the planning system, the physician and dosimetrist can outline key anatomical features, such as the tumor and organs at risk. After the outlines are drawn, radiation beams are placed at various angles around the target (tumor), and the beams are shaped based on the patient and tumor anatomy. Once the beams have been constructed, the treatment planning system calculates and displays the doses in three dimensions. Before the plan is finalized and delivered to the patient, it is approved by the physician. If an unsatisfactory dose distribution is obtained, initial treatment plans can be altered by changing beam shapes, angles, and positions and by adding accessories until the ideal dose distribution is obtained. Some planning systems have the capability of shaping beams not just by changing the size or position of the beam portal, but also by changing the intensity of the beam in certain places of the beam portal. This type of beam shaping is called intensity modulation. The type of treatment delivered using this technology is called intensity modulated radiation therapy (IMRT).

When it comes to the delivery of the beam, the patient has to be positioned on the treatment coach in an exact same way as when the planning data was acquired. This is crucial since the location of the gantry and shape of the beam is planned based on pre-treatment images. This problem is even more complex if the organ of interest can move independently from more stable bony structures of the body, especially in the prostate and breast, which are highly deformable organs, and their movement is dependent on other rather uncontrollable anatomical and physiological constraints.

In these cases, a pre-treatment verification is done, which enables the practitioner to update and refine the treatment plan based on the current state of the patient. An ultrasound-based imaging system is desirable since it has a non-ionizing beam and is real-time. Furthermore, for prostate applications, the outlines of the prostate are mostly visible in ultrasound images. A couple of companies have product solutions that are based on ultrasound imaging. Nomos, Zmed, and Brain Lab have similar systems in which an ultrasound probe tracked and co-registered with a linear accelerator is used on each treatment day prior to radiation in order to update the treatment plan. The shortcoming of this approach is that it does not account for deformation of the imaged organ. This approach assumes that only rigid movement happens in between the planning stage and the treatment stage. Furthermore, no automatic approach has been devised to align the daily images to the pre-treatment planning ones. As the result, the process of manual re-adjustment of the plan could be inaccurate and time-consuming.

### SUMMARY

The present invention is defined by the following claims, and nothing in this section should be taken as a limitation on those claims.

By way of introduction, the below embodiments relate to a method and system for modifying a radiation therapy treatment plan. In one embodiment, a radiation therapy treatment plan based on a first medical diagnostic image of a target in a patient is provided. A second medical diagnostic image of the target is generated while the patient is disposed on a treatment surface of a radiation therapy device. A deformation of the target is determined using the second medical diagnostic image, and the radiation therapy treatment plan is modified based on the determined deformation. Other embodiments are provided, and each of the embodiments described herein can be used alone or in combination with one another.

The embodiments will now be described with reference to the attached drawings.

### BRIEF DESCRIPTION OF THE DRAWINGS

FIG. 1 is a block diagram of a system for modifying a radiation therapy treatment plan of a preferred embodiment.

FIG. 2 is a flow chart of a method for modifying a radiation therapy treatment plan of a preferred embodiment.

FIG. 3 is a flow chart of another method for modifying a radiation therapy treatment plan of a preferred embodiment.

### DETAILED DESCRIPTION OF THE PRESENTLY PREFERRED EMBODIMENTS

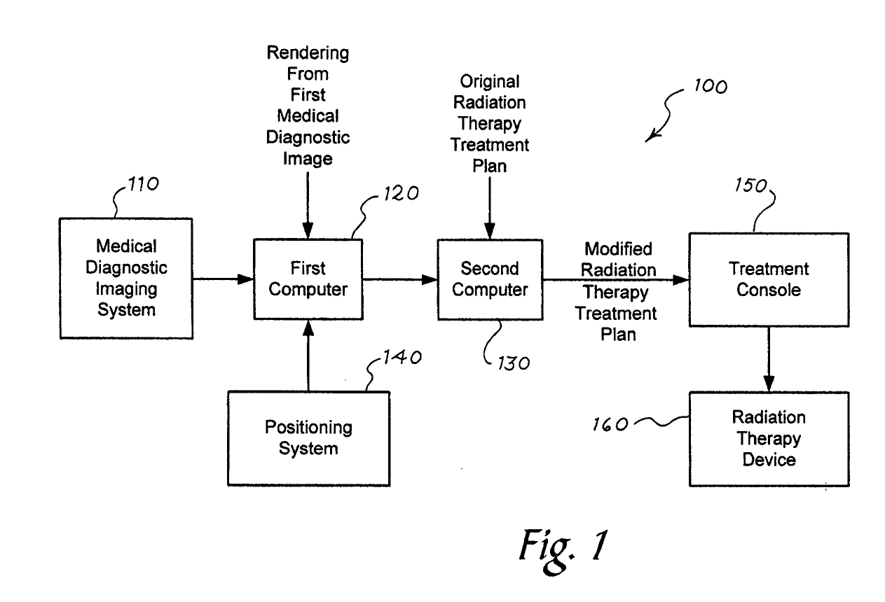

Turning now to the drawings, FIG. 1 is a block diagram of a system 100 for modifying a radiation therapy treatment plan of a preferred embodiment. A “radiation therapy treatment plan” describes how much radiation is needed (the dose of radiation) and how it should be delivered. The system 100 in FIG. 1 comprises a medical diagnostic imaging system 110, first and second computers 120, 130, a positioning system 140, a treatment console 150, and a radiation therapy device 160. In general, the radiation therapy device 160 comprises a treatment surface upon which a patient can be disposed and components to deliver radiation to a target in the patient according to the radiation therapy treatment plan (e.g., a linear accelerator, a collimator, a gantry). Examples of suitable radiation therapy devices 160 include, but are not limited to, the PRIMUS and ONCOR linear accelerators (“LINACs”) by Siemens Medical Solutions. The treatment console 150 is used to present a radiation therapy treatment plan to a physician before it is delivered to a patient.

The medical diagnostic imaging system 110 can be a stand-alone machine separate from the radiation therapy device 160 or can be physically integrated with the radiation therapy device 160. The medical diagnostic imaging system 110 generates a medical diagnostic image of a patent disposed on the treatment surface of the radiation therapy device 160. As used herein, the phrase “medical diagnostic image” refers to an image of anatomy taken using any suitable imaging modality, including, but not limited to, ultrasound, computed tomography (CT), magnetic resonance imaging (MRI), positron emission tomography (PET), computed radiography, and magnetic resonance.

In one preferred embodiment, the medical diagnostic imaging system 110 is an ultrasound imaging system comprising a transducer probe, a beamformer, a processor, and a display device. In operation, a sonographer contacts the transducer probe with a patient, and the ultrasound system generates an ultrasound image. The ultrasound system's processor causes the beamformer to apply a voltage to the transducer to cause it to vibrate and emit an ultrasonic beam into the portion of the patient's body in contact with the transducer. Ultrasonic energy reflected from the patient's body impinges on the transducer, and the resulting voltages created by the transducer are received by the beamformer. The processor processes the sensed voltages to create an ultrasound image frame, which is displayed on the display device, stored, or transmitted to other components. The ultrasound system can generate three-dimensional images using a three-dimensional transducer probe or by stacking together a plurality of two-dimensional images generated using a two-dimensional transducer probe that is tracked so that its geometric orientation is known.

The positioning system 140 tracks the probe of the medical diagnostic imaging system 110 so that the probe's position with respect to the coordinate system of the radiation therapy device 160 is known. Any suitable positioning system can be used. For example, the orientation of the probe can be determined by LEDs, ultrasonic emitters, or magnetic sensors on the probe. By aligning the probe with the coordinate system of the radiation therapy device 160, the position and orientation of the probe can be determined with respect to the radiation therapy device 160. This provides a known orientation of the medical diagnostic images generated by the imaging system 110.

The computers 120, 130 can be general-purpose or application-specific computers. Each computer 120, 130 can comprise a processor and a computer-readable storage medium comprising computer-readable program code (i.e., software) stored therein that is executed by the processor. Alternatively, a purely hardware implementation can be used. It is important to note that while two computers 120, 130 are shown in FIG. 1, the functionality described below with respect to these computers 120, 130 can be distributed in the system 100 in any suitable manner. For example, a single computer can be used instead of two separate computers 120, 130. Also, the functionally describe below with respect to one or more of the computers 120, 130 can be implemented in another component of the system 100 (e.g., the treatment console 150 or the medical diagnostic imaging system 110).

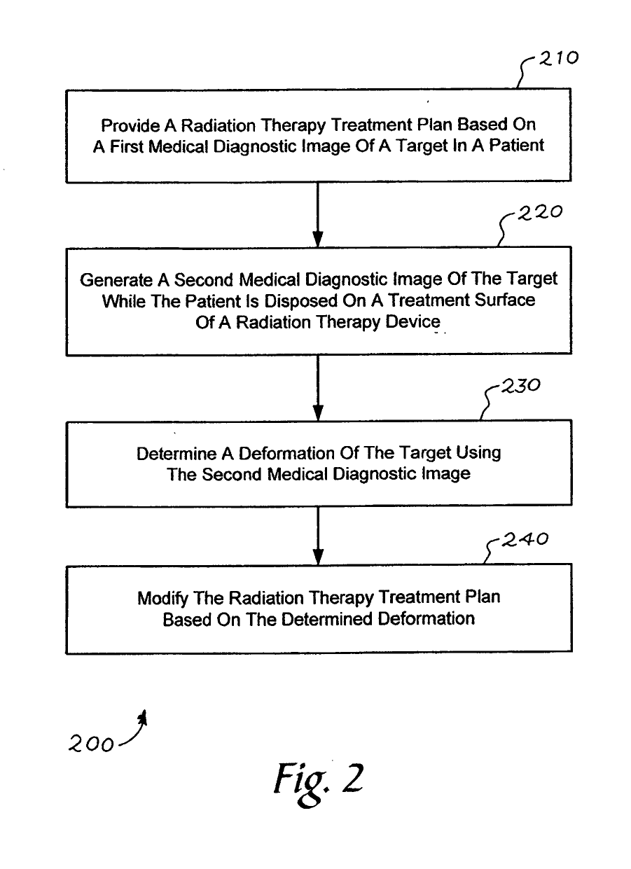

Turning again to the drawings, FIG. 2 is a flow chart 200 of a method for modifying a radiation therapy treatment plan of a preferred embodiment. First, a radiation therapy treatment plan based on a first medical diagnostic image of a target in a patient is provided to the first computer 120 (act 210). An image can preferably be either a bi-dimensional array of pixels or a three-dimensional array of voxels (i.e., volumetric image). The first medical diagnostic image is generated prior to treatment and will be referred to herein as a “planning image.” Next, while the patient is disposed on a treatment surface of the radiation therapy device 160, a second medical diagnostic image of the target is generated with the medical diagnostic imaging system 110 (act 220). The second medical diagnostic image is generated during the treatment session (i.e., generated “intra-treatment”) and will be referred to herein as a “localization image.” The first medical diagnostic image can be of a different type than the second medical diagnostic image (such as when the first medical diagnostic image is a computer tomography (CT) image, and the second medical diagnostic image is an ultrasound image). Alternatively, the first and second medical diagnostic images can be of the same type. Preferably, the second medical diagnostic image is a three-dimensional image (e.g., generated with a three-dimensional probe or a tracked two-dimensional probe).

The second medical diagnostic image is provided to the first computer 120 along with positioning information from the positioning system 140. The first computer also receives a rendering from the first medical diagnostic image, which was used to create the original radiation therapy treatment plan. A “rendering” can be created by outlining the target an image. A three-dimensional rendering can be created by outlining the target in each of a plurality of image slices and connecting a set of points together to form a “mesh.” The first computer 120 uses the second medical diagnostic image (with the positioning information) and the rendering from the first medical diagnostic image to determine a deformation of the target (act 230). As used herein, “deformation of a target” refers to a change in shape of the target—not merely a rigid movement of the target.

The determined deformation and the original radiation therapy plan are provided to the second computer 130, which modifies the original radiation therapy treatment plan based on the determined deformation (act 240). Modifications to the radiation therapy treatment plan can include, but are not limited to, changing at least one of a radiation therapy beam shape, angle, position, or intensity; adding an accessory to the radiation therapy device 160; and updating a multi-leaf collimator configuration (for IMRT). The second computer 130 sends the modified radiation therapy plan to the treatment console 150 for review by the physician. Upon approval by the physician, the modified radiation therapy plan is executed by the radiation therapy device 160.

This method provides an advantage over the approaches described above in the background section, which are concerned only with rigid movement of a target. As described in the background section, it is important to position the patient on the treatment surface such that the target is in the position expected by the radiation therapy treatment plan. Prior systems have generated two orthogonal localization ultrasound images of a target while a patient is on a treatment surface of a radiation therapy device. The physician overlays the ultrasound images onto the treatment plan and align them in a manual fashion. Based on the results of this comparison, the treatment surface can be moved to position the patient so that that target is in the location expected by the radiation therapy treatment plan. The problem with this approach is that it only takes into account rigid movement of the target and not deformation of the target. If the target grew smaller, for example, more radiation than necessary would be applied to the patient, jeopardizing tissue surrounding the target (“organs at risk”). By taking into account the deformation of a target and not merely the rigid movement of the target, a radiation therapy treatment plan can be more accurately modified to protect the organs at risk. In other words, by knowing the deformation of the target, the system 100 can calculate more than just a patient offset—it can re-adapt the radiation therapy treatment plan to the new deformed anatomy.

A description of a presently preferred ultrasound-based patient positioning method for radiation therapy will now be presented. In general, this method performs patient positioning and dose-delivery-plan refinement using ultrasound imaging. Daily ultrasound images from a three-dimensional probe or tracked two-dimensional probe prior to treatment are used to refine and update the treatment plan, which is usually outlined days or weeks prior to the treatment and is generated using planning CT images. The co-registration of the daily localization ultrasound images (e.g., both two dimensional and three dimensional) with the treatment plan can be achieved by segmenting the common structures in both modalities. Aligning these structures considering both rigid and deformable displacement will result in registration of the full content of the mentioned data sets. The segmentation and registration are preferably done in a unified framework. The outcome is a deformation field, which can be used to update the pre-treatment plan and to recompute the daily dose delivery and refine and optimize the overall treatment strategy.

Multi-modal deformable image/volume registration is a well-researched topic. See, for example, Wells, M. W., Viola, et. al., “Multi-Modal Volume Registration by Maximization of Mutual Information,” Medical Image Analysis, volume 1, number 1, pp 35-51; and Huesman R. H., Klein G. J. et. al., “Deformable Registration of Multi-Modal Data Including Rigid Structures,” IEEE Trans. on Nuclear Science, 50, 3, 2003, both of which are hereby incorporated by reference herein. It is preferred to choose a well-behaved similarity measure and good parameterized deformation model that can robustly characterize local metric for the volumes and nature of the movement. Since ultrasound images are inherently noisy, the deformable registration problem becomes even harder to solve. The driving force of most deformable registration algorithms is local information, which is more or less overwhelmed by ultrasound speckle noise patterns.

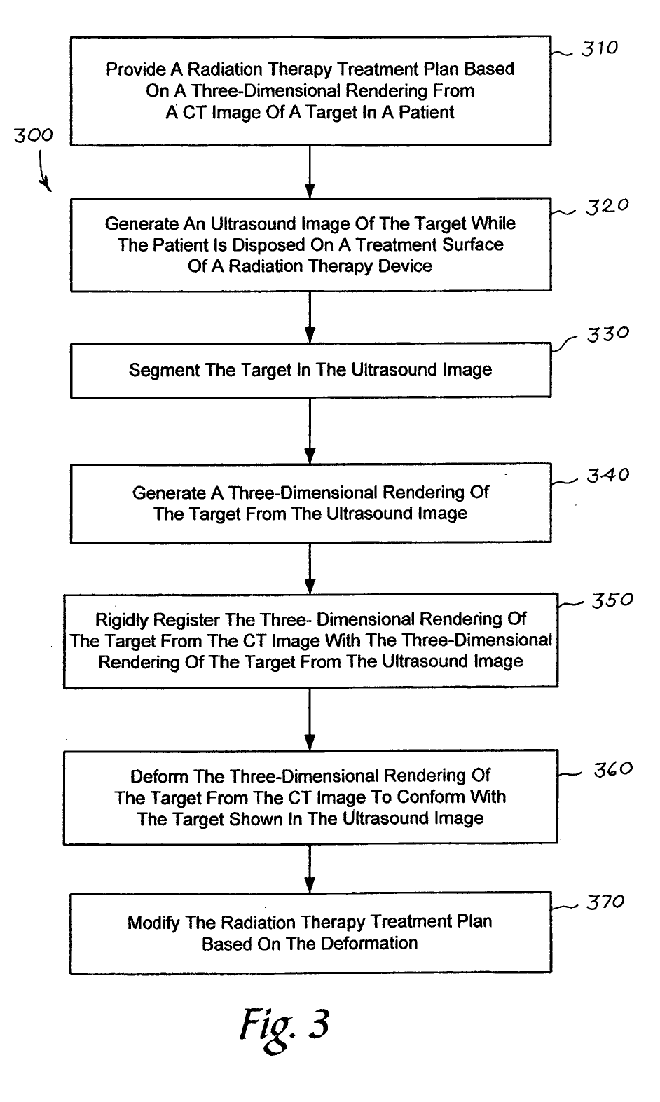

Returning to the drawings, FIG. 3 is a flowchart of a method of this embodiment. As shown in FIG. 3, a radiation therapy treatment plan based on a three-dimensional rendering from a planning CT image of a target in a patient is provided (act 310). Next, a localization ultrasound image of the target is generated while the patient is disposed on a treatment surface of a radiation therapy device (and 320). Preferably, the localization ultrasound image is either volumetric at its origin or is compounded using tracking information. It is also preferred that the localization ultrasound image coordinates are known in linear-accelerator iso-centric coordinate frame.

Next, the target in the localization ultrasound image is roughly segmented (i.e., the target is outlined in the image) (act 330). The target can be segmented manually, automatically, or semi-automatically. A three-dimensional rendering of the target from the localization ultrasound image is then generated (act 340). The rendering can be a contour of a set of points on the outline(s) generated on the three-dimensional image (or on each of the two-dimensional images). Next, the three-dimensional rendering of the target from the localization ultrasound image is rigidly registered with the three-dimensional rendering of the target from the planning CT image (i.e., renderings from the outlines of the target on the pretreatment planning images) (act 350). The three-dimensional renderings can be rigidly registered automatically and can be rigidly registered using an iterative closed point (ICP)-based algorithm. One suitable ICP-based algorithm is described in P. J. Besl, N. D. McKay, “A Method for Registration of 3-D Shapes,” Proc. of IEEE Transactions on Pattern Analysis and Machine Intelligence (PAMI), vol. 14 no. 2, pp. 239-256, 1992, which is hereby incorporated by reference.

The three-dimensional rendering of the target from the planning CT image is then deformed to conform with the target shown in the localization ultrasound image (act 360). By overlaying the outlines of the plan onto the localization ultrasound images, the content of the localization ultrasound image is used as the driving force to refine the segmentation and align the planned contours to the real borders of the organ. After this refinement step, the dense displacement vectors that map the pre-treatment plan to the current localization ultrasound imaging state are found. There are some constraints that one might consider. These may include constraining the deformation map to one that has a realizable inverse or to parameterize the deformation model to smaller, more manageable degrees of freedom.

The deformation can be performed using a parameterized deformable model, as described in T. F. Cootes, C. J. Taylor, D. H. Cooper, and J. Graham, “Active Shape Models—Their Training and Application,” Computer Vision and Image Understanding, 61 (1):38-59, January 1995, which is incorporated herein by reference. Also, the deformation can comprise overlaying a segmentation of the three-dimensional rendering from the planning CT image onto the localization ultrasound image and automatically refining the segmentation of the three-dimensional rendering from the planning CT image to match an outline of a corresponding structure in the localization ultrasound image. The segmentation can be refined using a level-set based segmentation method, a fluid-based deformation model, an elastic-based deformation model, or a deformation model with less than three hundred (or three thousand) degrees of freedom. Level-set based segmentation methods are describe in S. Osher and J. Sethian. “Fronts Propagating with Curvature-Dependent Speed: Algorithms Based on Hamilton-Jacobi Formulations,” Journal of Computational Physics, 79, pp 12-49, 1988; and L. A. Vese and T. F. Chan, “A Multiphase Level Set Framework for Image Segmentation Using the Mumford and Shah Model,” The International Journal of Computer Vision, 50 (3):271-293, 2002, both of which are hereby incorporated by reference. Also, a dense deformation field based on a deformation field acquired by matching contours of the three-dimensional rendering of the target from the planning CT image to edges of the target in the localization ultrasound image can be computed. The dense deformation field can be constrained to have an inverse. Also, the deformation of corresponding contours from the three-dimensional rendering from the planning CT image and the three-dimensional rendering from the localization ultrasound image can be propagated to an enclosed volume using a finite element method based on physical properties of the target.

The registration technique described above combines segmentation and registration in a unified framework in order to find free-form displacement between the updated localization ultrasound images and the pre-treatment plan. By knowing the displacements among the pre-treatment plan coordinates and the ultrasound coordinates, the radiation therapy treatment plan can be modified based on the deformation to update the plan according to the current state of patient (act 370).

It is intended that the foregoing detailed description be understood as an illustration of selected forms that the invention can take and not as a definition of the invention. It is only the following claims, including all equivalents, that are intended to define the scope of this invention.

1. A method for modifying a radiation therapy treatment plan, the method comprising:
(a) providing a radiation therapy treatment plan based on a first medical diagnostic image of a target in a patient;
(b) generating a second medical diagnostic image of the target while the patient is disposed on a treatment surface of a radiation therapy device;
(c) determining a deformation of the target using the second medical diagnostic image; and
(d) modifying the radiation therapy treatment plan based on the determined deformation.

2. The method of claim 1, wherein the first medical diagnostic image comprises a computer tomography (CT) image, and wherein the second medical diagnostic image comprises an ultrasound image.

3. The method of claim 1, wherein the first medical diagnostic image is of a same type as the second medical diagnostic image.

4. The method of claim 1, wherein the first medical diagnostic image is of a different type than the second medical diagnostic image.

5. The method of claim 1, wherein the radiation therapy treatment plan is based on a rendering of the target from the first medical diagnostic image, and wherein the method further comprises:
generating a rendering of the target from the second medical diagnostic image; and
rigidly registering the rendering of the target from the first medical diagnostic image with the rendering of the target from the second medical diagnostic image.

6. The method of claim 5 further comprising:
deforming the rendering of the target from the first medical diagnostic image to conform with the target shown in the second medical diagnostic image.

7. The method of claim 1, wherein (d) comprises changing at least one of a radiation therapy beam shape, angle, position, or intensity.

8. The method of claim 1, wherein (d) comprises adding an accessory to the radiation therapy device.

9. The method of claim 1, wherein (d) comprises updating a multi-leaf collimator configuration.

10. A computer-readable storage medium comprising computer-readable program-code stored therein for modifying a radiation therapy treatment plan based on a first medical diagnostic image of a target in a patient, the computer-readable program code comprising:
first computer-readable program code operative to determine a deformation of the target using a second medical diagnostic image of the target generated while the patient is disposed on a treatment surface of a radiation therapy device; and
second computer-readable program code operative to modify the radiation therapy treatment plan based on the determined deformation.

11. The computer-readable storage medium of claim 10, wherein the first medical diagnostic image comprises a computer tomography (CT) image, and wherein the second medical diagnostic image comprises an ultrasound image.

12. The computer-readable storage medium of claim 10, wherein the first medical diagnostic image is of a same type as the second medical diagnostic image.

13. The computer-readable storage medium of claim 10, wherein the first medical diagnostic image is of a different type than the second medical diagnostic image.

14. The computer-readable storage medium of claim 10, wherein the radiation therapy treatment plan is based on a rendering of the target from the first medical diagnostic image, and further comprising:
third computer-readable program code operative to generate a rendering of the target from the second medical diagnostic image;
fourth computer-readable program code operative to rigidly register the rendering of the target from the first medical diagnostic image with the rendering of the target from the second medical diagnostic image; and
fifth computer-readable program code operative to deform the rendering of the target from the first medical diagnostic image to conform with the target shown in the second medical diagnostic image.

15. A system for modifying a radiation therapy treatment plan based on a first medical diagnostic image of a target in a patient, the system comprising:
means for generating a second medical diagnostic image of the target while the patient is disposed on a treatment surface of a radiation therapy device;
means for determining a deformation of the target using the second medical diagnostic image; and
means for modifying the radiation therapy treatment plan based on the determined deformation.

16. The system of claim 15, wherein the first medical diagnostic image comprises a computer tomography (CT) image, and wherein the second medical diagnostic image comprises an ultrasound image.

17. The system of claim 15, wherein the first medical diagnostic image is of a same type as the second medical diagnostic image.

18. The system of claim 15, wherein the first medical diagnostic image is of a different type than the second medical diagnostic image.

19. The system of claim 15, wherein the radiation therapy treatment plan is based on a rendering of the target from the first medical diagnostic image, and wherein the system further comprises:
means for generating a rendering of the target from the second medical diagnostic image; and
means for rigidly registering the rendering of the target from the first medical diagnostic image with the rendering of the target from the second medical diagnostic image.

20. The system of claim 19 further comprising:
means for deforming the rendering of the target from the first medical diagnostic image to conform with the target shown in the second medical diagnostic image.

21. A method for modifying a radiation therapy treatment plan, the method comprising:
(a) providing a radiation therapy treatment plan based on a three-dimensional rendering from a computed tomography (CT) image of a target in a patient;
(b) generating an ultrasound image of the target while the patient is disposed on a treatment surface of a radiation therapy device;
(c) segmenting the target in the ultrasound image;
(d) generating a three-dimensional rendering of the target from the ultrasound image;
(e) rigidly registering the three-dimensional rendering of the target from the CT image with the three-dimensional rendering of the target from the ultrasound image;
(f) deforming the three-dimensional rendering of the target from the CT image to conform with the target shown in the ultrasound image; and
(g) modifying the radiation therapy treatment plan based on the deformation.

22. The method of claim 21, wherein the ultrasound image comprises a three-dimensional ultrasound image generated using a three-dimensional probe.

23. The method of claim 21, wherein the ultrasound image comprises a three-dimensional ultrasound image generated from tracked two-dimensional ultrasound images.

24. The method of claim 21, wherein (c) comprises automatically segmenting the target in the ultrasound image.

25. The method of claim 21, wherein (c) comprises semi-automatically segmenting the target in the ultrasound image.

26. The method of claim 21, wherein the three-dimensional rendering of the target from the CT image and the three-dimensional rendering of the target from the ultrasound image are rigidly registered in (e) automatically.

27. The method of claim 21, wherein the three-dimensional rendering of the target from the CT image and the three-dimensional rendering of the target from the ultrasound image are rigidly registered in (e) using an iterative closed point (ICP)-based algorithm.

28. The method of claim 21, wherein (f) is performed using a parameterized deformable model.

29. The method of claim 21, wherein (f) comprises:
overlaying a segmentation of the three-dimensional rendering from the CT image onto the ultrasound image; and
automatically refining the segmentation of the three-dimensional rendering from the CT to match an outline of a corresponding structure in the ultrasound image.

30. The method of claim 29, wherein the segmentation is refined using a level-set based segmentation method.

31. The method of claim 29, wherein the segmentation is refined using a fluid-based deformation model.

32. The method of claim 29, wherein the segmentation is refined using an elastic-based deformation model.

33. The method of claim 29, wherein the segmentation is refined using a deformation model with less than three hundred degrees of freedom.

34. The method of claim 29, wherein the segmentation is refined using a deformation model with less than three thousand degrees of freedom.

35. The method of claim 21, wherein (f) comprises computing a dense deformation field based on a deformation field acquired by matching contours of the three-dimensional rendering of the target from the CT image to edges of the target in the ultrasound image.

36. The method of claim 35, wherein the dense deformation field is constrained to have an inverse.

37. The method of claim 21 further comprising:
propagating deformation of corresponding contours from the three-dimensional rendering from the CT image and the three-dimensional rendering from the ultrasound image to an enclosed volume using a finite element method based on physical properties of the target.

38. The method of claim 21, wherein (g) comprises changing at least one of a radiation therapy beam shape, angle, position, or intensity.

39. The method of claim 21, wherein (g) comprises adding an accessory to the radiation therapy device.

40. The method of claim 21, wherein (g) comprises updating a multi-leaf collimator configuration.
